(19) United States
(12) Patent Application Publication
(10) Pub. No.: US 2012/0028910 A1
(43) Pub. Date: Feb. 2, 2012

(54) STORAGE-STABLE AQUEOUS OPHTHALMIC FORMULATIONS

(76) Inventors: Jean-Philippe Combal, Dijon (FR); Elisabeth Latour, Paris (FR); Harun Takruri, Newport Beach, CA (US)

(21) Appl. No.: 12/681,982

(22) PCT Filed: Oct. 8, 2008
(86) PCT No.: PCT/EP08/08482

§ 371 Date: Oct. 21, 2011

(30) Foreign Application Priority Data
Oct. 8, 2007 (EP) 07360047.0

(51) Int. Cl. A61K 38/13; A61P 29/00; A61P 27/02

(52) U.S. Cl. 514/20.5

(57) ABSTRACT

Storage-stable, topically ocularly administrable aqueous ophthalmic formulations containing at least one closporine are useful for treating and/or preventing ophthalmic diseases or disorders afflicting humans or animals, notably related to inflammatory conditions, more particularly administrable to the front of the eye and which provide therapeutic effects to the eye as they are effective in stabilizing, enhancing and/or improving a patient's vision.

The present invention relates to ophthalmic formulations and methods for treating and/or preventing ophthalmic diseases or disorders of humans or animals. The present ophthalmic formulations and methods are highly suitable for ocular administration, more particularly administration in the front of the eye, and provide therapeutic effects to the eye as they are effective in stabilizing, enhancing and/or improving a patient's vision. More specifically, the present invention relates to ophthalmic formulations and methods for preventing and/or treating ophthalmic diseases or disorders directly and/or indirectly related to inflammatory conditions.

Ophthalmic diseases or disorders in general terms are conditions which affect the eye or one of the regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball. They can be divided into:

(i) front-of-the-eye (FOE) diseases or disorders which affect anterior regions of the eye, such as periocular muscle, eye lid or eyeball tissue or fluid located anterior to the posterior wall of the lens capsule or ciliary muscles. Thus, front-of-eye diseases or disorders primarily concern the conjunctiva, the cornea, the anterior chamber, the iris, the posterior chamber (behind the retina but in front of the posterior wall of the lens capsule), the lens or the lens capsule and blood vessels and nerve which vascularize or innervate an anterior ocular region. Examples of front-of-eye diseases or disorders are anterior uveitis, allergy, aphakia, pseudophakia, astigmatism, blepharospasm, cataract, conjunctival diseases, conjunctivitis (including allergic conjunctivitis), corneal diseases, corneal diseases or opacifications with an exudative or inflammatory component, corneal oedema, corneal ulcer, dry eye syndromes, eyelid diseases, lacrimal apparatus diseases, lacrimal duct obstruction, laser induced exudation, myopia, presbyopia, pterygium, pupil disorders, refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure);

and (ii) back-of-the-eye (BOE) diseases or disorders which affect posterior regions of the eye, such as choroid or sclera, vitreous, vitreous chamber, retina, optic nerve, and blood vessels and nerves which vascularize or innervate a posterior ocular region. Examples of back-of-eye diseases or disorders are choroidal neovascularization; acute macular neuroretinopathy; exudative eye diseases and more particularly Behcet's disease, exudative retinopathies, macular degeneration (such as non-exudative age related macular degeneration and exudative age related macular degeneration); macular oedema, retinal disorders, diabetic retinopathy, retinopathy of prematurity, retinal arterial occlusive disease; central retinal vein occlusion; uveitis (including intermediate and anterior uveitis); retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma. Glaucoma can be considered a posterior ocular condition because a therapeutic goal can be to prevent the loss of or reduce the occurrence of loss of vision due to damage to or loss of retinal cells or optic nerve cells (i.e. neuroprotection).

Inflammation of the eye may be localized to the eye, the eyes, or may be part of more generalized inflammatory process. Its etiology may be infection, allergy, immunological reactions, or as a response to surgery, injury, or due to any other causes. The ocular inflammation causes pain, irritation, watering, threatens visual function of the eye and may also change optical properties of the eye. The ocular inflammatory diseases include uveitis, conjunctivitis (including allergic conjunctivitis), cyclitis, scleritis, episcleritis, optic neuritis, retrobulbar optic neuritis, keratitis, blepharitis, corneal ulcer, conjunctival ulcer, and extend to ocular diseases which while not being directly inflammatory disorders are a consequence of said inflammation (e.g. oedemas, retinopathies, etc . . . ).

Further, the ocular inflammatory diseases may be caused by various ocular disorders, an ophthalmic operation or a physical injury to the eye.

The symptoms of the ocular inflammatory diseases include itching, flare, oedema, ulcer, etc. The patients with ocular inflammatory diseases account for more than half of all the patients with ocular diseases. Accordingly, agents having ocular anti-inflammatory effects play an important role in the medical care. Today, steroid and non-steroidal drugs are mainly used for the ocular inflammatory diseases. The steroid drugs, which have excellent effects on the ocular inflammatory diseases, are clinically indispensable drugs. However, whether they are administered systemically or topically, they have the risk of bringing serious side effects. Such side effects include, for example, steroid glaucoma, infectious eye diseases, steroid cataract, etc. Especially, patients with chronic ocular inflammatory diseases have a high risk of such side effects. Additionally, for the specific patients having an already increased intraocular pressure (e.g. glaucoma patients), such side effects can never be acceptable

Under these circumstances, it has been strongly desired to develop alternative to the currently available therapeutic strategies. One applicant's strategy is to use lower doses of corticosteroids which can achieve the same or better therapeutic effects as those observed with larger doses of corticosteroids compositions. Such lower doses may be realized with the said compositions because they are containing special adjuvant and thus may exhibit greater therapeutic activity as compared to the equivalent composition without said adjuvant, which means that smaller doses of corticosteroids are likely required to obtain the desired therapeutic effect. Actually, the Applicant has surprisingly found that cyclosporines were capable to improve the therapeutic effect raised by corticosteroid towards ocular pathologies, and more specifically were capable to design therapeutic protocols where the corticosteroid dose administered are below therapeutic values.

Cyclosporines are a group of nonpolar cyclic oligopeptides, which have a broad spectrum of useful pharmacological activities, particularly immuno-suppressive activity and anti-inflammatory activity. The major cyclosporine metabolite is cyclosporine A.

Cyclosporine inhibits T cell activation and causes suppression of cell-mediated immune response. Cyclosporine has been used for suppression of immunological responses caused by tissue and organ transplantation, for example transplantation of the heart, lung, liver, kidney, pancreas, bone marrow, skin and cornea, and especially the transplantation of foreign tissues and organs. In addition, cyclosporine is useful for the suppression of hematological disorders such as anemia, various autoimmune diseases such as systemic lupus erythematosus and idiopathic malabsorption syndrome, and inflammatory diseases such as arthritis and rheumatoid disorders. Cyclosporine is also useful in ophthalmology, such as for example in treating patients with dry eye syndrome (see Wilson and Perry, 2007, Ophthalmology, 114, 6-9), progressive vascularising keratitis in keratitis-ichthyosis-deafness (KID) syndrome (Senter syndrome) (Derse et al., 2002, Klin Monatsbl Augenheilkd, 219, 383-386), steroid-resistant atopic keratoconjunctivitis (Akpek et al., 2004, Ophthalmology, 111, 476-482), or posterior segment intraocular inflammation (Murphy et al, 2005, Arch Ophthalmol, 123, 634-641).

Cyclosporines of natural origin which in their majority comprise cyclosporine A and in their minority the cyclosporines B to I can be obtained from the fungus Trichoderma polysporum, however like a large number of their analogs and isomers, cyclosporines can also be obtained by synthesis. The cyclosporine most widely studied and used in pharmacy among the cyclosporines is cyclosporine A.

Nevertheless, cyclosporine is a neutral, highly lipophilic and hydrophobic, cyclic endecapeptide with a molecular weight of 1200 daltons. More specifically, cyclosporine has a low aqueous solubility (e.g. 20 to 40 μg/mL for cyclosporine A measured at 25° C., Ran et al., 2001, AAPS PharmSci Tech, 2(1), Article 2 ; Akhlaghi and Trull, 2002, Clin. Pharmacokinet, 41, 615-637) and readily precipitates in the presence of water (e.g. on contact with body fluids), while it is well dissolved in organic solvents such as methanol, ethanol, acetone, ether, chloroform, DMSO, DMF and the like. However, these solvents are not compatible with ophthalmic uses. Thus, cyclosporine is very difficult to formulate into ophthalmic composition due to its low water solubility, and this special solubility issue needs to be resolved in order to prepare combination products with corticoids, including those as above mentioned.

Numerous studies have been extensively conducted to discover preparations suitable for the effective ocular administration of cyclosporine, which can provide a suitable uniform dosage and appropriate bioavailability. It is for this reason that cyclosporine, known to be lipophilic, has mainly been used in the prior art in oil-based formulations.

Patent U.S. Pat. No. 4,839,342 describes a topical ophthalmic formulation containing a cyclosporine, particularly cyclosporine A, and an excipient which can be selected in the group consisting of olive oil, peanut oil, castor oil, polyethoxylated castor oil, mineral oils, vaselines, dimethyl sulfoxide, an alcohol, liposomes, silicone oils or their mixtures.

Patent application FR 2,638,089 describes a topical ophthalmic formulation which contains a cyclosporine as the active substance and a vegetable oil such as olive oil, peanut oil, castor oil, sesame oil and maize germ oil as the vehicle, as well as vaseline, to treat conditions affecting the eye (e.g. keratoconjunctivitis sicca (KCS) or dry eyes).

Patent application EP 0760237 describes a pre-concentrate microemulsion composition comprising a water insoluble pharmaceutically active material such as cyclosporine, a C8-C20 fatty acid mono-, di- or tri glyceride from a vegetable oil or any mixture of two or more thereof, a phospholipid and another surfactant.

Methods of providing cyclosporine formulations with improved bioavailability are further described in U.S. Pat. No. 4,388,307, U.S. Pat. No. 6,468,968, U.S. Pat. No. 5,051,402, U.S. Pat. No. 5,342,625, U.S. Pat. No. 5,977,066, and U.S. Pat. No. 6,022,852.

However, the oil-based ophthalmic formulations have disadvantages such as low eye tolerance, irritation and oils may moreover reinforce undesirable symptoms of the disease, such as for example inflammation or dry-eye symptoms. For the purposes of minimizing these disadvantages WO 95/31211 proposes to reduce the amount of oil and disperse the oil phase in water so as to form an emulsion, which gave a topical ophthalmic formulation in the form of an emulsion based on water and on oil comprising a cyclosporine mixed with a triglyceride containing long-chain fatty acids such as castor oil and polysorbate 80. Cyclosporine microemulsion compositions are further described in U.S. Pat. No. 5,866,159, U.S. Pat. No. 5,916,589, U.S. Pat. No. 5,962,014, U.S. Pat. No. 5,962,017, U.S. Pat. No. 6,007,840, and U.S. Pat. No. 6,024,978.

Nevertheless, oil-based topical ophthalmic formulations containing cyclosporine are physically unstable, and are not adapted for ocular administration.

U.S. Pat. No. 5,951,971 discloses an aqueous topical ophthalmic formulation which is free of oil and comprises a cyclosporine in a concentration of 0.01 to 0.075% (w/v), water, and a surfactant in an amount of 0.1 to 3% (w/v) intended to improve the solubility of the cyclosporine in water and selected among the polyethoxylated fatty acid esters, the polyethoxylated alkylphenyl ethers, the polyethoxylated alkyl ethers and their mixtures. Similarly, Kuwano et al.(2002, Pharmaceutical Research 19, 108-111) propose to use the surfactant polyoxyl 40 stearate (MYS-40) in order to improve the solubility of the cyclosporine in water and to prepare formulation able to deliver therapeutic levels of cyclosporine. Additionally in U.S. Pat. No. 5,951,971, it has been found that Tween 80 (i.e. polysorbate 80) is inappropriate as a surfactant, because it lacks an activity sufficiently high to solubilise a cyclosporine in the desired concentrations in water.

Patent application US 2004106546 suggests to use Tween combined with hyaluronic acid for preparing an aqueous topical ophthalmic formulation containing cyclosporine, said hyaluronic acid permitting to solubilise the cyclosporine while improving the bioavailability of the formulation in the conjunctiva, cornea, and lachrymal gland and eye tolerance of the formulation.

Therefore, there is still a need for new aqueous ophthalmic formulation containing cyclosporine, which will not contain oil or derivates, or organic solvents of a type or at concentrations that are not safe or suitable for ophthalmic administration.

One first objective of the Invention was to provide, such an aqueous formulation, usable in ophthalmology among other uses, which is safe and suitable for topical ocular, periocular and intraocular administration, does not contain oil or organic solvent of a type or at concentrations that are not safe.

Another objective of the Invention was to provide such an aqueous formulation further containing corticosteroids, and more specifically low doses of corticosteroids.

Another objective of the present Invention was to provide methods for preventing and/or treating ophthalmic diseases or disorders directly and/or indirectly related to inflammatory conditions.

Another objective of the present Invention was to provide methods for enhancing the ocular pharmacological efficacy of a corticosteroid in a patient. This method comprises providing a pharmaceutical formulation containing at least one cyclosporine with an amount of at least one corticosteroid wherein said amount is such that it provides a reduced pharmacological activity in the absence of said cyclosporine.

More specifically, the objective of the present invention was to provide an aqueous pharmaceutical formulation for preventing and/or treating ocular conditions, comprising a cyclosporine and a corticosteroid as active ingredients. Even more specifically, the objective of the present invention was to provide an aqueous pharmaceutical formulation for preventing and/or treating front-of-eye conditions, comprising a cyclosporine and a corticosteroid as active ingredients. Due to the difference in the physicochemical properties and chemical stability profiles of these active ingredients, especially cyclosporine (see above), it was a challenge to develop a stable, active, safe formulation combining these drugs. Furthermore, pharmaceutical formulations of water-insoluble drugs in aqueous medium for ocular, as well as other uses, must satisfy constraints imposed by physiological compatibilities such as pH, osmolarity, and particle size of the suspended drug if any.

As used herein throughout the entire application, the terms “a” and “an” are used in the sense that they mean “at least one”, “at least a first”, “one or more” or “a plurality” of the referenced compounds or steps, unless the context dictates otherwise. More specifically, “at least one” and “one or more” means a number which is one or greater than one, with a special preference for one, two or three.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. “About x %” also encompasses x specific number.

As used herein, the term “comprising”, “containing” when used to define products, formulations and methods, is intended to mean that the products, formulations and methods include the referenced compounds or steps, but not excluding others.

It was the aim of the present invention to provide a water-based ophthalmic formulation, more specifically a topical formulation, containing a cyclosporine which obviates the above listed disadvantages, including the problems of physical stability and where cyclosporine is in solution without the addition of oil or any unsafe organic solvent, that is stable, and where cyclosporine ocular bioavailability and/or tolerance are not compromised. The inventors have now found that the presence of surface active agent in combination with nonionic tonicity agent in an aqueous ophthalmic formulation containing cyclosporine surprisingly permitted to solubilise the cyclosporine while improving the bioavailability of the formulation in the conjunctiva, cornea, and lachrymal gland and eye tolerance of the formulation when this formulation is administered topically to the eyes.

According to a first embodiment, the Invention provides an aqueous formulation comprising (a) at least one cyclosporine; (b) a surface active agent and (c) nonionic tonicity agent wherein the cyclosporine solubility in the said formulation is above about 20 μg/mL at about 25° C.

According to one special embodiment, the Invention provides an aqueous formulation comprising (a) at least one cyclosporine; (b) a surface active agent and (c) nonionic tonicity agent wherein the cyclosporine solubility in the said formulation is between about 20 μg/mL and 40 μg/mL at about 25° C.

According to one preferred embodiment, the Invention provides an aqueous formulation comprising (a) at least one cyclosporine; (b) a surface active agent and (c) nonionic tonicity agent wherein the cyclosporine solubility in the said formulation is above 40 μg/mL at about 25° C.

According to another embodiment, the aqueous formulation of the present invention is further comprising (d) at least buffering agents wherein the cyclosporine solubility in the said formulation is above about 20 μg/mL and wherein the pH of the aqueous formulation is stable for at least 3 months, preferably 9 months, more preferably 12 months, and even preferably 24 months.

According to another preferred embodiment, the aqueous formulation of the present invention is further comprising (d) at least buffering agents wherein the cyclosporine solubility in the said formulation is above 40 μg/mL and wherein the pH of the aqueous formulation is stable for at least 3 months, preferably 9 months, more preferably 12 months, and even preferably 24 months.

According to another embodiment, the aqueous formulation of the present invention is stable for at least 3 months, preferably 9 months, more preferably 12 months, and even preferably 24 months. “Aqueous formulation of the present invention is stable” means that after 3, 9, 12 or 24 months at a selected temperature (preferably at about 25° C.) the amount of cyclosporine present in the aqueous formulation of the present Invention is reduced from a maximum of 10%, preferably a maximum of 5%, compared to the amount present initially after preparation of the aqueous formulation, preferably after filtration step if any. According to specific embodiments, the said stability can be improved by storing the formulation of the Invention at temperature below 10° C., more specifically between about 2° C. and about 8° C.

According to advantageous conditions, the aqueous ophthalmic formulations of the Invention are stored at temperature comprised between about 2° C.-8° C. and about 15° C.-25° C. Alternatively, the formulations of the invention are stored at 2° C.-8° C. for a certain period of time and at temperature between 15° C.-25° C. for another period.

According to preferred embodiment, when the aqueous ophthalmic formulations of the Invention is containing about 0.02% of cyclosporine, it is advantageously stored at temperature comprised between about 2° C. and about 8° C.

According to preferred embodiment, when the aqueous ophthalmic formulations of the Invention is containing about 0.01% of cyclosporine, it is advantageously stored at about 25° C.

In the present application, the term “cyclosporine” is to be understood to include whatever individual member of the class of cyclosporines and their mixtures, unless a particular cyclosporine is specified. The cyclosporines that may be contained in the formulation of the present invention can be of natural or synthetic origin. According to a preferred embodiment, the cyclosporine contained in the formulation is cyclosporine A. According to special embodiment said cyclosporine is an analogue of cyclosporine such as the one disclosed in patent application US 20070087963. Cyclosporine A is commercially available for example under the trade name Neoral™ (Novartis). Cyclosporine A structural and functional analogs include cyclosporines having one or more fluorinated amino acids (e.g. U.S. Pat. No. 5,227,467); cyclosporines having modified amino acids (e.g. U.S. Pat. No. 5,122,511 and U.S. Pat. No. 4,798,823); and deuterated cyclosporines, such as ISAtx247 (see patent application US 20020132763). Additional cyclosporine analogs are described in U.S. Pat. No. 6,136,357, U.S. Pat. No. 4,384,996, U.S. Pat. No. 5,284,826, and U.S. Pat. No. 5,709,797. Cyclosporine analogs include, but are not limited to, D-Sar ([alpha]-SMe)<3> Val<2>-DH-Cs (209-825), Allo-Thr-2-Cs, Norvaline-2-Cs, D-Ala(3-acetylamino)-8-Cs, Thr-2-Cs, and D-MeSer-3-Cs, D-Ser(O—CH₂CH₂—OH)-8-Cs, and D-Ser-8-Cs, which are described in Cruz et al. (2000, Antimicrob. Agents Chemother. 44, 143-149).

According to one specific embodiment, said surface active agent (b) is acceptable for ophthalmic uses and is non-ionic.

According to another specific embodiment, said surface active agent (b) is selected in the group consisting of polysorbates, poloxamers (e.g. poly(oxypropylene)-poly(oxyethylene) copolymer, Pluronic F-68), tyloxapol and lecithin.

According to another specific embodiment, said surface active agent (b) is selected in the group consisting of polysorbate 20 (PS20), polysorbate 40 (PS40), polysorbate 60 (PS60), and in preferred embodiment is polysorbate 80 (PS80).

According to another specific embodiment, said nonionic tonicity agent (c) is selected in the group consisting of low molecular weight hydrophilic polymers, propylene glycol, glycerin, sorbitol, mannitol and similar carbohydrates.

According to another specific embodiment, said nonionic tonicity agent (c) is a low molecular weight hydrophilic polymer and more particularly is selected from the group consisting of polyethylene glycols PEG (e.g. PEG 200, PEG 300, PEG 400, PEG 600), hydrophilic peptides, polysaccharides, polyethylene oxides. Preferably the tonicity agent is a polyethylene glycol, and preferably it is PEG 300.

According to special embodiment, the Invention provides an aqueous formulation comprising or consisting of (a) at least one cyclosporine; (b) polysorbate 80 and (c) PEG 300.

According to another specific embodiment, said buffering agent (d) is present and is selected in the group consisting of acetates, citrates, phosphates, and borates or other ophthalmologically acceptable buffers. According to preferred embodiments, the buffering agent is selected in order to maintain pH of the aqueous formulation between about 4 and about 7.5, preferably between between about 5 and about 7 for at least 3 months, 6 months, 9 months, 12 months, 24 months at max about 25° C. In preferred embodiment, the buffering agent is selected in order to maintain pH of the aqueous formulation between about 5 and about 6.5 for at least 3 months, 6 months, 9 months, 12 months, 24 months at max about 25° C.

The aqueous formulation according to the present invention preferably comprises between about 0.004% to about 0,1%, preferably between about 0,004% to about 0.05%, by weight of cyclosporine based on the formulation's total weight. According to special embodiments, the effective amount of cyclosporine is between about 0.001% and about 0.049% (e.g., 0.04%, 0.03%, 0.02%, 0.01%, 0.009%, 0.008%, 0.007%, 0.006%, and 0.005%). In preferred embodiment it is about 0.02% or less, and in even preferred embodiment it is about 0.01%.

Advantageously, the concentration of component (b) is about 0.01 to about 5% by weight, based on the aqueous formulation's total weight, in preferred embodiment, the formulation according to the present invention preferably comprises less than about 0.5% by weight of component (b). In special embodiment, the aqueous formulation according to the present invention preferably comprises about 0.2% to about 0.3% by weight of component (b).

The aqueous formulation according to the present invention preferably comprises less than about 9% by weight of PEG300 (compound c) based on the formulation's total weight. In preferred embodiment, the aqueous formulation according to the present invention preferably comprises 7% by weight of PEG300.

According to alternative embodiments, equivalent molar amounts of PEG 200, 400, or 600 might be used.

According to another specific embodiment, said buffering agent (d) is present in the aqueous formulation of the invention and is comprised between about 0.1% and about 0.5% (e.g. w/v for citric acid).

According to another embodiment, the aqueous formulation of the invention is further comprising (e) at least one corticosteroid. According to one preferred embodiment, said corticosteroid (e) is present in a sub-therapeutically effective amount, and said cyclosporine (a) is present in an effective amount capable of increasing the pharmacological efficacy conferred by said sub-therapeutically effective amount corticosteroid relative to the same amount of corticosteroid with no cyclosporine.

According to the present invention, the term “corticosteroid” refers to any naturally occurring or synthetic compound characterized by a hydrogenated cyclopentanoperhydro-phenanthrene ring system and having immunosuppressive and/or antiinflammatory activity. Naturally occurring corticosteroids are generally produced by the adrenal cortex. Synthetic corticosteroids may be halogenated.

Non limiting examples of corticosteroids are l′-alpha, 17-alpha, 21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione;

17-alpha-hydroxypregn-4-ene-3,20-dione; 17-alpha-hydroxypregnenolone; 17-hydroxy-l6-beta-methyl-5-beta-pregn-9(11)-ene-3,20-dione; 17-hydroxy-4,6,8(14)-pregnatriene-3,20-dione; 17-hydroxypregna-4,9(11)-di- ene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxypredniso1one; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21 (beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

According to preferred embodiment, said corticosteroid is prednisolone, preferably prednisolone acetate or prednisolone sodium phosphate.

As used herein, a “sub-therapeutically effective amount of at least one corticosteroid” is defined as an amount that provides reduced or no pharmacological efficacy, more specifically reduced or no anti-inflammatory activity and/or anti-allergic activity, in the absence of any adjuvant, and more specifically in absence of cyclosporine. This reduced or lack of efficacy is observed in the absence of the cyclosporine while the same or about the same amount of the corticosteroid does demonstrate pharmacological efficacy in the presence of cyclosporine. In this regard the phenomenon is observed that the combination of low amounts of corticosteroids with cyclosporine have potent pharmacological efficacy (e.g. potent anti-inflammatory pharmacologic response) while doses of the drug alone (i.e. without cyclosporine) do not.

According to the present invention, the sub-therapeutically effective amount of a special corticosteroid is below the lowest approved concentration for ophthalmic administration of the said corticosteroid.

According to the present invention, the sub-therapeutically effective amount of a corticosteroid is typically from about 0.01% to about 4%, more particularly it is present in an amount of about 0.01% to about 1.0% (e.g., 1.0%, 0.9%, 0.8%, 0.7%, 0.6%, 0.5%, 0.1%, 0.09%, 0.08%, 0.07%, 0.06%, 0.05%, and 0.01%). In special embodiment it is in an amount of about 0.01% to about 0.12%.

Recommended dosages for Corticosteroid Dosages are as follows:

| | Lowest approved | |
|---|---|---|
| | concentration | Lowest standard |
| Ophthalmic | for ophthalmic | recommended |
| corticosteroid | administration | dosage |
| Clocortolone Pivalate | 0.1% | N/A |
| Hydrocortisone | 1.0% | 0.5 μg/3 |
| | | times daily |
| Dexamethasone | 0.1% | 0.05 μg/4-6 |
| | | times daily |
| Fluorometholone | 0.1% | 0.05 μg/2-4 |
| | | times daily |
| Loteprednol Etabonate | 0.2% | 0.1 μg/4 |
| | | times daily |
| Medrysone | 1.0% | 0.5 μg/up to |
| | | every 4 hours |
| Prednisolone Acetate | 0.12% | 0.06 μg/2-4 |
| | | times daily |
| Rimexolone | 1.0% | 0.5 μg/4 |
| | | times daily |

(N/A = Not Available)

Other standard recommended dosages for corticosteroids are provided, e.g., in the Merck Manual of Diagnosis & Therapy (17th Ed. M H Beers et al., Merck & Co.) and Physicians' Desk

Reference 2003 (57^{th} Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone.

According to the present invention, the sub-therapeutically effective amount of one corticosteroid can be either the lowest approved concentration of the said corticosteroid (see table above), or preferably 95% or less of the lowest approved concentration of the said corticosteroid. For example, low concentration of corticosteroids of the invention can be 90%, 85%, 80%, 70%, 60%, 50%, 25%, 10%, 5%, 2%, 1%, 0.5% or 0.1% of the lowest approved concentration.

For ophthalmic administration for example, a low concentration w/v of clocortolone pivalate is between 0.01% and 0.1% (e.g., 0.1%, 0.09%, 0.08%, 0.07%, 0.06%, 0.05%, and 0.01%), a low concentration of hydrocortisone is between 0.01% and 1.0% (e.g., 1.0%, 0.9%, 0.8%, 0.7%, 0.6%, 0.5%, 0.1%, 0.09%, 0.08%, 0.07%, 0.06%, 0.05%, and 0.01%), a low concentration of dexamethasone is between 0.01% and 0.1% (e.g., 0.1%, 0.09%, 0.08%, 0.07%, 0.06%, 0.05%, and 0.01%), a low concentration of fluorometholone is between 0.01% and 0.1% (e.g., 0.1%, 0.09%, 0.08%, 0.07%, 0.06%, 0.05%, and 0.01%), a low concentration of loteprednol etabonate is between 0.01% and 0.2% (e.g., 0.2%, 0.1%, 0.09%, 0.08%, 0.07%, 0.06%, 0.05%, and 0.01%), a low concentration of medrysone is between 0.01% and 1.0% (e.g., 1.0%, 0.9%, 0.8%, 0.7%, 0.6%, 0.5%, 0.1%, 0.09%, 0.08%, 0.07%, 0.06%, 0.05%, and 0.01%), a low concentration of rimexolone is between 0.01% and 1.0% (e.g., 1.0%, 0.9%, 0.8%, 0.7%, 0.6%, 0.5%, 0.1%, 0.09%, 0.08%, 0.07%, 0.06%, 0.05%, and 0.01%), and a low concentration of prednisolone acetate is between 0.01% and 0.12% (e.g., 0.12%, 0.1%, 0.09%, 0.08%, 0.07%, 0.06%, 0.05%, and 0.01%).

According to one special embodiment, the aqueous formulation of the Invention contains 0.12% w/v of prednisolone acetate and 0.02% w/v of cyclosporine.

According to another special embodiment, the aqueous formulation of the Invention contains less than 0.12% w/v of prednisolone acetate and less than 0.02% w/v of cyclosporine.

According to another special embodiment, the aqueous formulation of the Invention contains 0.02% w/v or less of cyclosporine.

According to one special embodiment, the aqueous formulation of the Invention contains 0.12% w/v of prednisolone acetate and 0.01% w/v of cyclosporine.

According to another special embodiment, the aqueous formulation of the Invention contains less than 0.12% w/v of prednisolone acetate and less than 0.01% w/v of cyclosporine.

According to one special embodiment, the aqueous formulation of the Invention contains 0.024% w/v of prednisolone acetate and 0.01% w/v of cyclosporine.

According to another special embodiment, the aqueous formulation of the Invention contains less than 0.024% w/v of prednisolone acetate and less than 0.01% w/v of cyclosporine.

According to another special embodiment, the aqueous formulation of the Invention contains 0.01% w/v or less of cyclosporine.

According to another embodiment, the aqueous formulation of the invention is further comprising (f) a suspending agent. Said suspending agent (f) is a water soluble polymer which allows the active drug particles to be suspended and preferably to remain suspended for a suitable time. Said suspending agent (f) can be selected from the group consisting of gelatin, alginate, chitosan, poly(methyl methacrylate), carbomers, water-soluble cellulose derivatives, polyvinyl alcohol, povidone, natural gums, hyaluronic acid, soluble starches.

According to one specific embodiment, said suspending agent (f) is a cellulose derivative such as methylcellulose, hydroxyethyl cellulose, hydroxypropyl cellulose, hydroxypropyl methylcellulose, hydroxypropyl ethylcellulose, carboxymethylcellulose.

In special embodiment said suspending agent (f) is cellulose derivate, and preferably it is hydroxyethyl cellulose (e.g. NatrosoirN, type 250 G-Pharm, Aqualon). According to alternative embodiment, other viscosity grades of hydroxyethyl cellulose might be used.

Said suspending agent (f) can be used in a concentration of about 0.05-5% w/v, about 0.2-2.5% w/v, and preferably about 0.3-1.75% w/v.

According to one embodiment, the pharmaceutical formulation of the invention is further comprising a preservative (g). Preferably, it is not interacting with the surface active agent to an extent that the preservatives are prevented from protecting the suspension from microbiological contamination. In a preferred embodiment benzalkonium chloride may be employed as a safe preservative, most preferably benzalkonium chloride with EDTA. Disodium edetate has also been found to be effective in reducing microbial growth in the present formulations. Other possible preservatives include but are not limited to benzyl alcohol, methyl parabens, propyl parabens, thimerosal, chlorbutanol and benzethonium chlorides. Preferably, a preservative (or combination of preservatives) that will impart standard antimicrobial activity to the suspension and protect against oxidation of components of the formulation is employed.

These preservatives are generally used in an amount of about 0.0001 to 0.5% w/v, and preferably about 0.001 to 0.015%. In special embodiment, the preservatives present in the pharmaceutical formulation of the invention are benzalkonium chloride and edetate disodium at 0.01% w/v and 0.01% w/v, respectively.

The pH of the aqueous formulations of the invention is preferably comprised between about 4 to about 8 (e.g. from about 4 to about 7.5), more preferably between about 4 to about 6.5.

The aqueous formulations of the invention provide novel pharmaceutical formulations containing water-insoluble drug suitable for therapeutic use. The invention provides stable aqueous formulations of (i) at least one cyclosporine in aqueous solution and (ii) at least one corticosteroid in aqueous solution or as a plurality of particles, wherein the mean particle sizes is less than about 15 μm, preferably less than about 10 μm, and advantageously less than about 5 μm which remain in such a state so as to allow for immediate suspension, when desired, even after extended periods of settling.

The aqueous formulations of the invention are suitable for therapeutic use in the eye. The aqueous formulation of the invention are surprisingly stable and can remain in a state suitable for immediate suspension when desired and if needed, even after extended periods of settling. The aqueous formulations of the invention, moreover, do not cause discomfort upon application.

The aqueous formulations of the invention are made by aseptic preparation. Purity levels of all materials employed in the formulation of the invention exceed 98%. The aqueous formulations of the invention are prepared by thoroughly mixing the active drugs (a) and (e), suspending agent, surface active agent, tonicity agents, buffering agents and preservatives if present.

The present invention further concerns process for the preparation of aqueous formulations of the Invention. Those skilled in the art recognize that each embodiment might require a different sequence for combining the various ingredients.

According to a first embodiment, said process comprises the following steps (Process A):

Preparation of Part I
the desired amount of surface active agent (b) and the desired amount of tonicity agent (c) (e.g. Polysorbate 80 and PEG 300, respectively) are combined and mixed to form homogeneous solution, at room temperature,
the desired amount of cyclosporine (a) is added and mixed until complete dissolution.

Preparation of Part II in parallel:
dissolve the buffering agent (d) and/or preservative (g) if any in purified water (about 80% of final formulation volume) at room temperature

Adjust pH to the designated value of the final formulation
Part I and Part II are pooled and mix to maintain homogeneity and complete solubility of cyclosporine.

pH is checked and readjusted if necessary.

Volume is adjusted to final with sterilized purified water under vigorous mixing and the final formulation mixed to homogeneity

Sterilisation by filtration is performed through a sterilizing filter (e.g. 0.2 μm) into a sterile vessel.

Sterile containers, preferably ophthalmic containers, are filled aseptically.

According to another embodiment, wherein the aqueous formulation is containing non water-soluble corticosteroid (e.g. prednisolone acetate), said process comprises the following steps (Process B):

Preparation of Part I
the desired amount of surface active agent (b) is mixed with the desired amount of tonicity agent (c)
the desired amount of cyclosporine (a) is added until complete dissolution of (a)

Preparation of Part II in parallel:
purified water (about 60% of final formulation volume) is heated to about 65-70° C.
the desired amount of suspending agent (f) is added and mixed to dissolution
the mixture is cooled to room temperature and the desired amount of preservatives (g) is added if any

Part I and Part II are pooled and the desired amount of buffering agent (d) is added and mixed; pH is adjusted and volume adjusted to about 90% of final volume with purified water; the preparation is mixed and pH adjusted again if necessary. The mixture is sterile-filtered using a suitable sterilizing filter and the desired amount of sterilized corticosteroid (e) is added to the mixture under vigorous mixing until complete and homogenous dispersion of the corticosteroid. Volume is adjusted to final with sterilized purified water and the final formulation mixed to homogeneity and filled aseptically in sterile containers, preferably ophthalmic containers.

Alternate sequences for the preparation of Part II might be utilized if necessary. For example the nonpolymeric ingredients might be dissolved first before heating the water and the addition of the polymer. Another method utilized especially when efficient high shear mixers are available is preparing Part II at room temperature without the aid of heat.

All or some of the other ingredients of Part II might be added before or after dissolving the polymer. Those skilled in the art recognize the variety of procedures that can be used to prepare Part II that result, when combined with Part I and the corticosteroid, in the same final product.

Alternate process for the preparation of the aqueous formulation containing water-insoluble corticosteroid (e.g. prednisolone acetate) might be utilized. Said process comprises the following steps (Process C):

Preparation of Part I:
the desired amount of surface active agent (b) is dissolved in purified water (about 85% of the final volume of Part I) then the solution is heated to about 65-70° C.
the desired amount of suspending agent (f) is added and mixed to dissolution; volume is adjusted to about 90% of final volume of Part I
the desired amount of corticosteroid (e) is added to the mixture under vigorous mixing, volume is adjusted to final and the final formulation is mixed to homogeneity and autoclaved (e.g. at 121° C. for lhour). The concentration of corticosteroid in this part can range from 2.5 to 20%, about 3-10%, and preferably 3-5%.

Preparation of Part IIA:
the desired amount of surface active agent (b) is mixed with the desired amount of tonicity agent(c)
the desired amount of cyclosporine (a) is added until complete dissolution of (a)

Preparation of Part IIB:
purified water (about 75% of final volume of Part II) is heated to about 65-70° C.
the desired amount of suspending agent (f) is added and mixed to dissolution
dissolve the preservative (g) and the buffering agent (d) and adjust pH to the designated value of the final formulation

Preparation of Part II: Part IIA and Part IIB are pooled and mixed. pH is checked and readjusted if necessary. Volume is adjusted to final with purified water. Sterilisation by filtration is performed through a sterilizing filter (e.g. 0.2 pm) into a sterile vessel.

Part I and Part II
Part I is shaken and added to Part II under gentle mixing and the final formulation is filled aseptically in sterile containers, preferably ophthalmic containers.

The present invention further concerns the aqueous formulations prepared according to the processes of the Invention.

In another aspect of the invention, the aqueous formulations of the invention may further comprise a compound selected in the group consisting of an oestrogen (e.g. oestrodiol), an androgen (e.g. testosterone) retinoic acid derivatives (e. g. 9-cis-retinoic acid, 13-trans-retinoic acid, all-trans retinoic acid), a vitamin D derivative (e.g. calcipotriol, calcipotriene), a non-steroidal anti-inflammatory agent, a selective serotonin reuptake inhibitor (SSR1; e.g. fluoxetine, sertraline, paroxetine), a tricyclic antidepressant (TCA; e.g. maprotiline, amoxapine), a phenoxy phenol (e.g. triclosan), an antihistaminine (e.g. loratadine, epinastine), a phosphodiesterase inhibitor (e.g. ibudilast), an anti-infective agent, a protein kinase C inhibitor, a MAP kinase inhibitor, an anti-apoptotic agent, a growth factor, a nutrient vitamin, an unsaturated fatty acid, and/or ocular anti-infective agents, for the treatment of the ophthalmic disorders set forth herein (see for example compounds disclosed in US 2003/0119786; WO 2004/073614; WO 2005/051293; US 2004/0220153; WO 2005/027839; WO 2005/037203; WO 03/0060026). In still other embodiments of the invention, a mixture of these agents may be used. Ocular anti-infective agents that may be used include, but are not limited to penicillins (ampicillin, aziocillin, carbenicillin, dicloxacillin, methicillin, nafcillin, oxacillin, penicillin G, piperacillin, and ticarcillin), cephalosporins (cefamandole, cefazolin, cefotaxime, cefsulodin, ceftazidime, ceftriaxone, cephalothin, and moxalactam), aminoglycosides (amikacin, gentamicin, netilmicin, tobramycin, and neomycin), miscellaneous agents such as aztreonam, bacitracin, ciprofloxacin, clindamycin, chloramphenicol, cotrimoxazole, fusidic acid, imipenem, metronidazole, teicoplanin, and vancomycin), antifungals (amphotericin B, clotrimazole, econazole, fluconazole, flucytosine, itraconazole, ketoconazole, miconazole, natamycin, oxiconazole, and terconazole), antivirals (acyclovir, ethyldeoxyuridine, foscarnet, ganciclovir, idoxuridine, trifluridine, vidarabine, and (S)-1-(3-dydroxy-2-phospho-nyluethoxypropyl) cytosine (HPMPC)), antineoplastic agents (cell cycle (phase) nonspecific agents such as alkylating agents (chlorambucil, cyclophosphamide, mechlorethamine, melphalan, and busulfan) anthracycline antibiotics (doxorubicin, daunomycin, and dactinomycin), cisplatin, and nitrosoureas), antimetabolites such as antipyrimidines (cytarabine, fluorouracil and azacytidine), antifolates (methotrexate), antipurines (mercaptopurine and thioguanine), bleomycin, vinca alkaloids (vincrisine and vinblastine), podophylotoxins (etoposide (VP-16)), and nitrosoureas (carmustine, (BCNU)), and inhibitors of proteolytic enzymes such as plasminogen activator inhibitors. Doses for topical and sub-conjunctival administration of the above agents, as well as intravitreal dose and vitreous half-life may be found in Intravitreal Surgery Principles and Practice, Peyman G A and Shulman, J Eds., 2nd edition, 1994, Appleton- Longe, the relevant sections of which are expressly incorporated by reference herein.

The aqueous formulations of the Invention are of particular interest for treating and/or preventing ocular pathologies.

According to another embodiment, the present invention relates to a method for inhibiting, treating, or preventing ocular diseases, and related disease or condition, in a patient in need of such treatment that comprises the step of administering an aqueous formulation of the present invention in said patient.

The term “patient” refers to a vertebrate, particularly a member of the mammalian species and includes, but is not limited to, domestic animals, sport animals, primates including humans. The term “patient” is in no way limited to a special disease status, it encompasses both patients who have already developed a disease of interest and patients who are not sick.

As used herein, the term “treatment” or “treating” encompasses prophylaxis and/or therapy. Accordingly the formulations and methods of the present invention are not limited to therapeutic applications and can be used in prophylaxis ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Among the ophthalmic disorders which can be treated or addressed in accordance with the present invention include, without limitation, exudative and/or inflammatory ophthalmic disorders. According to preferred embodiment, the ophthalmic disorders which can be treated according to the present invention are front-of-the-eye diseases or disorders, i.e. which affect anterior regions of the eye, such as periocular muscle, eye lid or eyeball tissue or fluid located anterior to the posterior wall of the lens capsule or ciliary muscles. Thus, front-of-the-eye diseases or disorders primarily concern the conjunctiva, the cornea, the anterior chamber, the iris, the posterior chamber (behind the retina but in front of the posterior wall of the lens capsule), the lens or the lens capsule and blood vessels and nerve which vascularize or innervate an anterior ocular region. Examples of front-of-the-eye diseases or disorders are anterior uveitis, allergy, aphakia, pseudophakia, astigmatism, blepharospasm, cataract, conjunctival diseases, conjunctivitis (including allergic conjunctivitis), corneal diseases, corneal diseases or opacifications with an exudative or inflammatory component, corneal oedema, corneal ulcer, dry eye syndromes, eyelid diseases, lacrimal apparatus diseases, lacrimal duct obstruction, laser induced exudation, myopia, presbyopia, pterygium, pupil disorders, refractive disorders and strabismus, ocular inflammatory disease caused by bacterial or viral infection, and by an ophthalmic operation, an ocular inflammatory disease caused by a physical injury to the eye, a symptom caused by an ocular inflammatory disease including itching, flare, edema and ulcer, erythema, erythema exsudativum multiforme, erythema nodosum, erythema annulare, scleredema, dermatitis, angioneurotic edema, laryngeal edema, glottic edema, subglottic laryngitis, bronchitis, rhinitis, pharyngitis, sinusitis, laryngitis or otitis media. Glaucoma can also be considered to be an anterior ocular condition because a clinical goal of glaucoma treatment can be to reduce a hypertension of aqueous fluid in the anterior chamber of the eye (i.e. reduce intraocular pressure).

Administration of the pharmaceutical formulations of the invention is preferably topical, although other modes of administration may be effective. Preferably, the ophthalmic formulations are administered in unit dosage forms suitable for single administration of precise dosage amounts.

The skilled reader will appreciate that the duration over which any of the pharmaceutical formulations used in the method of the invention will depend on such factors as the physicochemical and/or pharmacological properties of the compounds employed in the formulation, the concentration of the compound employed, the disease to be treated, the mode of administration and the preferred longevity of the treatment. Where that balance is struck will often depend on the longevity of the effect required in the eye and the ailment being treated.

The frequency of treatment according to the method of the invention is determined according to the disease being treated, the deliverable concentration of the active compounds. The frequency of dosage may also be determined by observation, with the dosage being delivered when the previously delivered pharmaceutical formulation is visibly cleared. In general, an effective amount of the compound is that which provides either subjective relief of symptoms or an objectively identifiable improvement as noted by the clinician or other qualified observer.

Pharmaceutical formulation prepared for used in the method of the present invention to prevent or treat ophthalmic disorders will preferably have dwell times from hours to many months and possibly years, although the latter time period requires special delivery systems to attain such duration and/or alternatively requires repetitive administrations. Most preferably the pharmaceutical formulation for use in the method of the invention will have a dwell time (i.e. duration in the eye) of hours (i.e. 1 to 24 hours), days (i.e. 1, 2, 3, 4, 5, 6 or 7 days) or weeks (i.e. 1, 2, 3, 4 weeks). Alternatively, the pharmaceutical formulation will have a dwell time of at least a few months such as, 1 month, 2 months, 3 months, with dwell times of greater than 4, 5, 6, 7 to 12 months being achievable.

If desired, the method or use of the invention can be carried out alone, or in conjunction with one or more conventional therapeutic modalities (such as photodynamic therapy, laser surgery, laser photocoagulation or one or more biological or pharmaceutical treatments. These methods are well known from the skilled man in the art and widely disclosed in the literature). The use of multiple therapeutic approaches provides the patient with a broader based intervention. In one embodiment, the method of the invention can be preceded or followed by a surgical intervention. In another embodiment, it can be preceded or followed by photodynamic therapy, laser surgery, laser photocoagulation. Those skilled in the art can readily formulate appropriate therapy protocols and parameters which can be used.

The present Invention further concerns a method for improving the treatment of a patient which is undergoing one or more conventional treatment as listed above, which comprises co-treatment of said patient along with an aqueous formulation of the present invention.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

The present invention is not to be limited in scope by the specific embodiments described herein, which are intended for the purpose of exemplification only. Functionally equivalent products, formulations and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

### EXAMPLES

### Example 1

According to one embodiment the aqueous formulation of the present Invention is detailed below.

Said formulation has been prepared according to Process B

| Compounds | % w/v |
|---|---|
| Cyclosporine A | 0.02 |
| Prednisolone acetate, micronized | 0.12 |
| Polysorbate 80 | 0.30 |
| PEG 300 | 7.00 |
| Benzalkonium chloride | 0.01 |
| Edetate disodium | 0.01 |
| HEC 250 | 0.3 |
| Citric acid (monohydrate) | 0.15 |
| HCl/NaOH pH | 6.5 +/− 0.1 |
| Purified water qs | 100 |

### Example 2

Said formulation has been prepared according to Process A (see below):

| Compounds | % w/v |
|---|---|
| Cyclosporine A | 0.005 |
| Polysorbate 80 | 0.30 |
| PEG 300 | 7.00 |
| Benzalkonium chloride | 0.01 |
| Edetate disodium | 0.01 |
| Citric acid (monohydrate) | 0.15 |
| HCl/NaOH pH | 6.5 +/− 0.1 |
| Purified water qsp | 100 |

Part I

Polysorbate 80 and PEG 300 are combined and mixed to form homogeneous solution. Cyclosporine is added and mixed until completely dissolved.

Part II

Dissolve the remaining ingredients at room temperature in about 80 of the water in the batch.

Adjust pH to the designated value

Add Part 1 quantitatively and mix to maintain homogeneity and complete solubility of cyclosporine.

Check pH and readjust if necessary.

Add while mixing sufficient water to batch volume.

Aseptically filter batch through a sterilizing filter into a sterile vessel

Fill aseptically into sterile ophthalmic containers.

### Example 3

According to one embodiment the aqueous formulation of the present Invention is detailed below.

Said formulation has been prepared according to Process B

| Compounds | % w/v |
|---|---|
| Cyclosporine A | 0.01 |
| Prednisolone acetate, micronized | 0.024 |
| Polysorbate 80 | 0.30 |
| PEG 300 | 7.00 |
| Benzalkonium chloride | 0.01 |
| Edetate disodium | 0.01 |
| HEC 250 | 0.3 |
| Citric acid (monohydrate) | 0.15 |
| HCl/NaOH pH | 5.2 +/− 0.1 |
| Purified water qs | 100 |

### Example 4

Said formulation has been prepared according to Process B

| Compounds | % w/v |
|---|---|
| Cyclosporine A | 0.02 |
| Prednisolone acetate, micronized | 0.12 |
| Polysorbate 80 | 0.30 |
| PEG 300 | 7.00 |
| Benzalkonium chloride | 0.01 |
| Edetate disodium | 0.01 |
| HEC 250 | 0.3 |
| Citric acid (monohydrate) | 0.15 |
| HCl/NaOH pH | 5.2 +/− 0.1 |
| Purified water qs | 100 |

### Example 5

Said formulation has been prepared according to Process A (see above):

| Compounds | % w/v |
|---|---|
| Cyclosporine A | 0.005 |
| Polysorbate 80 | 0.30 |
| PEG 300 | 7.00 |
| Benzalkonium chloride | 0.01 |
| Edetate disodium | 0.01 |
| Citric acid (monohydrate) | 0.15 |
| HCl/NaOH pH | 5.2 +/− 0.1 |
| Purified water qsp | 100 |

### Example 6

### Stability Data

6.1. The table below shows stability data of cyclosporine A in the composition of example 3 initially, and at 6 months storage at 25° C. (40% relative humidity) and 2-8° C.

| | | Month 6 | Month 6 |
|---|---|---|---|
| | Initial | (25° C./40% RH) | (2-8° C.) |
| HPLC assay | 100 | 102 | 103.6 |
| (% initial) | | | |
| pH | 5.22 | 5.14 | 5.21 |

The results show that there was no significant change in cyclosporine A concentration during the storage period, demonstrating good stability. Moreover, there was no change in the formulation over the 6-month period with respect to physical appearance, pH or osmolality.

6.2. The table below shows stability data of cyclosporine A in the composition of example 4 initially, at 3 months storage at 25° C. (40% relative humidity) and at 9 months storage at 2-8° C.

| | | Month 3 | Month 9 |
|---|---|---|---|
| | Initial | (25° C./40% RH) | (2-8° C.) |
| HPLC assay | 100 | 104.6 | 98.6 |
| (% initial) | | | |
| pH | 5.31 | 5.29 | 5.30 |

The results show that there was no significant change, neither in cyclosporine A concentration nor in physical appearance, pH or osmolality during the storage periods, demonstrating good stability.

1.-29. (canceled)

30. A storage-stable, topically ocularly administrable aqueous ophthalmic formulation comprising (a) at least one cyclosporine; (b) a surface active agent which comprises a polysorbate and (c) a nonionic tonicity agent which comprises a low molecular weight hydrophilic polymer.

31. The ocularly administrable ophthalmic formulation as defined by claim 30, said aqueous formulation further comprising (d) one or more buffering agents and wherein the pH of the ophthalmic formulation is stable for at least 3 months.

32. The ocularly administrable ophthalmic formulation as defined by claim 30, said at least one cyclosporine comprising cyclosporine A.

33. The ocularly administrable ophthalmic formulation as defined by claim 30, said surface active agent (b) being selected from the group consisting of polysorbate 20, polysorbate 40, polysorbate 60 and polysorbate 80.

34. The ocularly administrable ophthalmic formulation as defined by claim 30, said surface active agent (b) comprising polysorbate 80.

35. The ocularly administrable ophthalmic formulation as defined by claim 30, said nonionic tonicity agent (c) comprising a polyehtylene glycol PEG selected from the group consisting of PEG 200, PEG 300, PEG 400 and PEG 600.

36. The ocularly administrable ophthalmic formulation as defined by claim 35, said nonionic tonicity agent (c) comprising polyehtylene glycol PEG 300.

37. The ocularly administrable ophthalmic formulation as defined by claim 30, said aqueous formulation comprising (a) at least one cyclosporine, (b) polysorbate 80 and (c) PEG 300.

38. The ocularly administrable ophthalmic formulation as defined by claim 30, said aqueous formulation comprising from 0.004% to 0.1% w/v of cyclosporine.

39. The ocularly administrable ophthalmic formulation as defined by claim 30, said aqueous formulation comprising from 0.004% to 0.5% w/v of cyclosporine.

40. The ocularly administrable ophthalmic formulation as defined by claim 30, said aqueous ophthalmic formulation comprising from 0.001% to 0.049% w/v or less of cyclosporine.

41. The ocularly administrable ophthalmic formulation as defined by claim 30, said aqueous formulation comprising 0.02% w/v or less of clyclosporine.

42. The ocularly administrable ophthalmic formulation as defined by claim 30, said aqueous formulation comprising 0.01% to about 5% by weight of component (b).

43. The ocularly administrable ophthalmic formulation as defined by claim 30, said aqueous formulation comprising less than 0.5% by weight of component (b).

44. The ocularly administrable ophthalmic formulation as defined by claim 30, said aqueous formulation comprising 0.2% to 0.3% by weight of component (b).

45. The ocularly administrable ophthalmic formulation as defined by claim 36, said aqueous formulation comprising less than 9% by weight of PEG 300.

46. The ocularly administrable ophthalmic formulation as defined by claim 36, said aqueous formulation comprising about 7% by weight of PEG 300.

47. The ocularly administrable ophthalmic formulation as defined by claim 30, said aqueous formulation comprising from 0.1% to 0.5% of buffering agent (d).

48. The ocularly administrable ophthalmic formulation as defined by claim 30, said aqueous formulation comprising (e) at least one corticosteroid.

49. The ocularly administrable ophthalmic formulation as defined by claim 48, said at least one corticosteroid being selected from the group consisting of 1′-alpha, 17-alpha, 21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-di-ene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21 (beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; wortmannin; and mixture thereof.

50. The ocularly administrable ophthalmic formulation as defined by claim 49, said at least one corticosteroid comprising prednisolone.

51. The ocularly administrable ophthalmic formulation as defined by claim 50, said at least one corticosteroid comprising prednisolone acetate or prednisolone sodium phosphate.

52. The ocularly administrable ophthalmic formulation as defined by claim 48, wherein the amount of said at least one corticosteroid ranges from 0.01% to 4%.

53. The ocularly administrable ophthalmic formulation as defined by claim 52, wherein the amount of said at least one corticosteroid ranges from 0.01% to 0.12%.

54. A process for preparing an ocularly administrable ophthalmic formulation as defined by claim 30, said process comprising the following steps:
(i) preparation of Part I
the desired amount of surface active agent (b) and the desired amount of tonicity agent (c) are combined and mixed to form a homogeneous solution, at room temperature,
the desired amount of cyclosporine (a) is added and mixed until complete dissolution;
(ii) preparation of Part II in parallel:
dissolve a buffering agent (d) and/or preservative (g) if any in purified water at room temperature;
(iii) adjust the pH to a designated value and combine Part I and Part II and mix to maintain homogeneity and complete solubility of the cyclosporine.

55. A regime or regimen for inhibiting, treating or preventing ocular diseases, and related disease or condition, in a patient in need of such treatment, comprising topically ocularly administering a thus effective amount of a ophthalmic formulation as defined by claim 30 to said patient.

56. The regime or regimen as defined by claim 55, said ocular disease and related disease or condition comprising an exudative and/or inflammatory ophthalmic disorder.

57. The regime or regimen as defined by claim 55, said ocular disease and related disease or condition comprising a front-of-the-eye disease or disorder.

58. The regime or regimen as defined by claim 57, wherein said front-of-the-eye disease or disorder is selected from the group consisting of uveitis, allergy, aphakia, pseudophakia, astigmatism, blepharospasm, cataract, conjunctival diseases, conjunctivitis, allergic conjunctivitis, corneal diseases, corneal diseases or opacifications with an exudative or inflammatory component, corneal oedema, corneal ulcer, dry eye syndromes, eyelid diseases, lacrimal apparatus diseases, lacrimal duct obstruction, laser induced exudation, myopia, presbyopia, pterygium, pupil disorders, refractive disorders and strabismus, ocular inflammatory disease caused by bacterial or viral infection, and by an ophthalmic operation, an ocular inflammatory disease caused by a physical injury to the eye, a symptom caused by an ocular inflammatory disease including itching, flare, edema and ulcer, erythema, erythema exsudativum multiforme, erythema nodosum, erythema annulare, scleredema, dermatitis, angioneurotic edema, laryngeal edema, glottic edema, subglottic laryngitis, bronchitis, rhinitis, pharyngitis, sinusitis, laryngitis, otitis media and/or glaucoma.
